(19) United States
(12) Patent Application Publication
(10) Pub. No.: US 2002/0189615 A1
(43) Pub. Date: Dec. 19, 2002

(54) Inhaler with dose control

(76) Inventors: Adam S. Henry, San Diego, CA (US); Robert F. Eisele, Carlsbad, CA (US); Thomas R. Jackson, La Jolla, CA (US); Karen R. Davies, Vista, CA (US); Dan Moore, San Diego, CA (US)

Correspondence Address: PERKINS COIE LLP, POST OFFICE BOX 1208, SEATTLE, WA, 98111-1208, US

(21) Appl. No.: 09/882,372

(22) Filed: Jun. 13, 2001

(51) Int. Cl. A61M 15/00

(52) U.S. Cl. 128/203.21

(57) ABSTRACT

A dry powder inhaler includes systems to prevent or inhibit inadvertent delivery of multiple doses. A latching or locking actuation button prevents closing the inhaler and release of a subsequent dose, until the first dose is inhaled. The actuation button is released, under control of an electronic controller linked to a sensor which senses inhalation. A latching system prevents closing the inhaler until the latching system is released, helping the patient to avoid inadvertently releasing multiple doses. The latching system may include a lockout, also controlled by an electronic controller or microprocessor, which prevents closing the inhaler until the released dose is inhaled. A visual warning system may also be provided to indicate a potential inadvertent multiple dose condition to the patient.

### BACKGROUND OF THE INVENTION

The field of this invention is inhalers. More specifically, the invention relates to dry powder inhalers for delivering a pharmaceutical in a solid finely divided dry powder or aerosol form.

Inhalers are used to delivery pharmaceuticals into a patient's lungs. Typically, an inhaler contains or provides a mixture of pharmaceuticals and air or propellant gases. The mixture is delivered with the patient inhaling from a mouthpiece on the inhaler, for treatment of lung diseases or conditions, by the presence of the pharmaceutical powder in the lung, or for treatment of other diseases or conditions, via systemic absorption of the pharmaceutical in the lungs.

One type of dry powder inhaler uses individual pharmaceutical doses sealed within blisters on a blister disk. The disk is advanced by mechanical movement, with each successive dose. See for example, U.S. Pat. Nos. 4,627,432, and 5,921,237 (Spiros Inhaler), and U.S. Pat. No. 4,811,731 (Diskhaler), incorporated herein by reference. Alternatively, a dry powder inhaler may operate with a strip of blisters, or on individual blisters loaded one at a time. Other inhalers meter out a dose of powder with each actuation. See for example, U.S. Pat. No. 5,829,434 (Turbuhaler). While dry powder inhalers of the type shown for example, in U.S. Pat. No. 5,921,237 perform well, they necessarily rely on patient compliance with operating instructions, to have the patient receive the correct dose. With this type of inhaler, a patient could inadvertently inhale the contents of two or more blisters, by failing to follow proper inhaler operating instructions. Alternatively, a patient could inadvertently fail to receive any dose, by activating the inhaler without first opening a blister on the blister disk and moving the dose of pharmaceutical powder from the blister into the inhaler.

Accordingly, it is an object of the invention to provide dry powder inhalers which reduce the potential for a patient to receive an incorrect dose.

### SUMMARY OF THE PREFERRED EMBODIMENT

In a first aspect of the invention, in a method for operating an inhaler, a dose of a pharmaceutical is loaded into a delivery path of the inhaler. A multi-dose deterrent is set or engaged. The deterrent remains engaged until the patient inhales the dose. The deterrent is then released or disengaged, to allow a subsequent dose to be loaded into the delivery path. The delivery path is preferably an air flow path in the inhaler. This method helps to prevent a patient from loading and inhaling more than one dose at a time. For pharmaceuticals where more than one dose should be inhaled at one time (i.e., doses should be accumulated before they are inhaled), the deterrent may optionally include a counter. The counter counts doses The deterrent is set or engaged after a predetermined number of doses are delivered into the delivery path, as counted by the counter.

In an inhaler designed to perform this method, the multi-dose deterrent may be mechanical, electromechanical, or electrically operated.

In a second aspect, in a method for operating an inhaler, a dose of a pharmaceutical is loaded into a delivery path A dose indicator is turned on. This indicates to the user or patient that a dose has been loaded. The indication may be made visually (e.g., a light is turned on), or via sound, tactile feel, etc. The indicator remains activated or on until the dose is inhaled. It is then turned off. This indicates to the patient that a subsequent may be loaded, if appropriate.

In an inhaler designed to perform this method, the dose indicator is preferably electrically controlled.

In a third and separate aspect, a dry powder inhaler for providing individual doses of a dry powder pharmaceutical formulation, from a blister disk, includes a blister disk advancing mechanism for incrementally advancing the blister disk. An actuator in the housing is movable from a first position, where the actuator does not engage the advancing mechanism or blister disk, to a second position where the actuator engages the advancing mechanism or the blister disk. The actuator is automatically held in the second position, to prevent the advancing mechanism or blister disk from moving (to position another blister for delivery), until a detecting system detects that the dose from the previous blister has been inhaled.

In a fourth aspect of the invention, the actuator is preferably held into the second position by an electromagnet controlled by an electronic controller linked to a breath actuated switch. As the blister disk advancing mechanism or the blister disk cannot be advanced until released by the actuator moving back into the second position, a patient cannot deliver a second dose from the blister disk, until the first dose is inhaled. Inhalation of the first dose is detected via the breath actuated switch, causing the controller to release the actuator from the advancing mechanism and/or the blister disk.

In a fifth aspect of the invention, the blister disk advancing mechanism includes a spinner assembly pivotably attached to a cover assembly on the inhaler housing. The spinner assembly is advantageously pivotable from a closed position, where the spinner assembly covers the mouthpiece of the inhaler, to an open position, where the spinner assembly is moved away and exposes the mouthpiece. Movement of the spinner assembly from the closed position to the open position, does not advance the blister disk, while movement of the spinner assembly from the open position to the closed position moves the blister disk to position another blister over a powder port in the housing. A button on the actuator is pushed in to open a blister on the blister disk, to release pharmaceutical powder into the powder port, and to move the actuator into the second position, where the actuator locks the spinner assembly from movement. A holder holds the actuator into the second position. Consequently, the inhaler cannot be returned to the closed position, with the spinner assembly covering the mouthpiece, until the dose of powder in the powder port is inhaled, causing the actuator to release from the spinner assembly.

In a sixth and separate aspect of the invention, a spinner assembly is pivotably attached to a cover assembly on a housing of an inhaler. The spinner assembly includes a projection or feature for engaging and advancing the blister disk, as the spinner assembly is moved from an open position to a closed position on the inhaler. A spinner latch on the spinner assembly engages with a housing latch on the housing, causing the spinner assembly to be locked into the open position, until the housing latch and spinner assembly latch are manually disengaged.

In a seventh and separate aspect of the invention, a spinner assembly is pivotably attached to a cover assembly on an inhaler housing. The spinner assembly advances a blister disk on the housing, by one position, when the spinner assembly is moved from an open position to a closed position. A housing latch engages a spinner assembly latch on the spinner assembly, locking the spinner assembly into the open position. A gate is moveable from a first position, where the gate prevents disengagement of the housing latch and the spinner assembly latch, to a second position where the gate is moved out of the way, to allow disengagement of the housing latch and spinner assembly latch. A gate actuator is attached to the gate. Preferably, an electronic controller within the inhaler is linked to the gate actuator and an inhalation detector. After a dose is released from a blister on the blister disk into the inhaler, the gate prevents movement of the spinner assembly, so that the inhaler cannot be closed, and no further doses can be delivered, until the first dose is inhaled. When the first dose is inhaled, the controller detects the inhalation via the inhalation detector, and actuates the gate actuator, to move the gate to the second position, thereby allowing the spinner assembly to be moved to the closed position. The gate actuator advantageously comprises a permanent magnet latching solenoid.

Other features and advantages, including methods of operation of a dry powder inhaler, will appear below.

The invention resides as well in subcombinations of the components, features, and steps described. Features and steps described in connection with one embodiment may of course also be included in other embodiments.

### BRIEF DESCRIPTION OF THE DRAWINGS

In the drawings, wherein the same reference number indicates the same element, throughout the several views:

FIG. 1 is a flow chart showing a first method of the invention.

FIG. 2 is a flow chart showing a second method of the invention.

FIGS. 3A and 3B are schematic drawing of an inhaler having multi-dose deterrent features.

FIG. 4 is a top and front perspective view of another inhaler embodiment, shown in the closed position.

FIG. 5 is a perspective view of the inhaler of FIG. 4, shown in the open position.

FIG. 6 is a bottom and front perspective view of the inhaler shown in FIG. 5.

FIG. 7 is a top perspective view of a blister disk for use in the inhaler shown in FIGS. 4-6.

FIG. 8A is a perspective view of the inhaler shown in FIGS. 4-6, with the cover assembly in an open position, for installing the blister disk shown in FIG. 7.

FIG. 8B is a perspective view of the inhaler as shown in FIG. 8A and further illustrating additional features.

FIG. 9 is a bottom and front perspective view, in part section, of the inhaler shown in FIGS. 4-7, 8A and 8B, with the actuation button shown in a first or released position, and with various components removed for clarity of illustration.

FIG. 10 is a view similar to FIG. 9, and showing the actuation button in a second or engaged position.

FIG. 11 is an enlarged partial section view of the actuation button shown in FIGS. 9 and 10.

FIG. 12 is top perspective view of the actuation button shown in FIGS. 9-11.

FIG. 13 is a top view of the housing of a third inhaler embodiment omitting the cover assembly and the spinner assembly on the cover assembly, for clarity of illustration.

FIG. 14 is a bottom view of an enclosure section of a spinner assembly of the embodiment shown in FIG. 13.

FIG. 15 is a bottom view, in part section, of a fourth inhaler embodiment.

FIG. 16 is an enlarged bottom view of the spinner and housing latches shown in FIG. 12.

FIG. 17 is a top view of a fifth inhaler embodiment, with the spinner assembly in the closed position.

FIG. 18 is a top view of the inhaler shown in FIG. 17, with the spinner assembly in the open position.

### DETAILED DESCRIPTION OF THE PREFERRED EMBODIMENTS

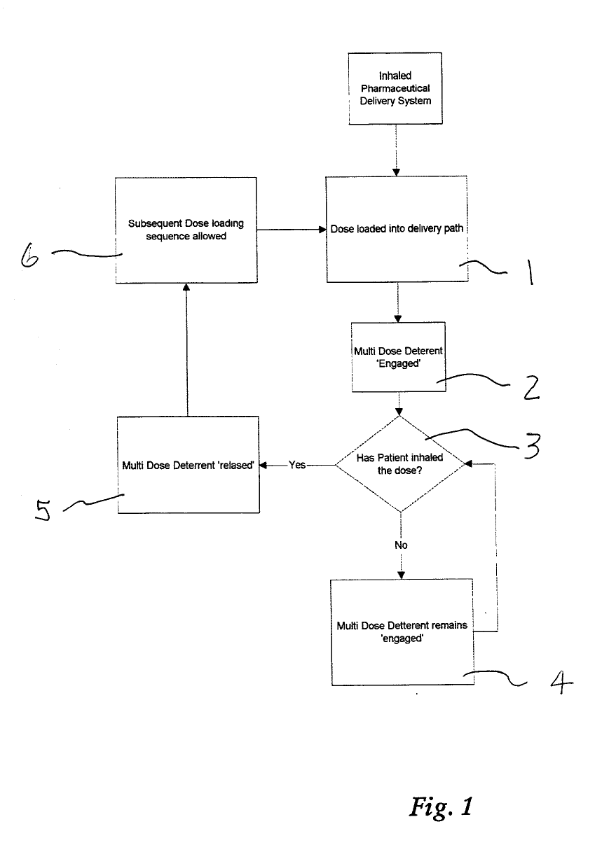

Turning now in detail to the drawings, as shown in FIG. 1, in a method for operating an inhaler or other pharmaceutical delivery system, at Step 1, a dose is loaded into a delivery path. This loading step may be performed by releasing a pharmaceutical preparation from an individual container, such a a blister, cassette well, capsule or similar container holding a single dose. This step may alternatively be performed by metering out a single dose from a bulk storage container or volume. Typically, the dose is a dry powder. The dose is released or otherwise moved, dropped, placed, etc., into a delivery path. The delivery path may be a chamber, conduit, or other temporary holding location (collectively referred to here as a delivery path). The delivery path is connected, at least indirectly, to a nose piece or mouthpiece, on which the patient or user inhales.

At Step 2, delivery of the dose into the delivery path is detected and a multi-dose deterrent is engaged. This prevents or discourages delivery of any further doses into the delivery path, until the first dose is inhaled. Detection of delivery of the dose into the delivery path may be made by sensing movement of components associated with delivery of the dose, such as a plunger, lever, piercing device, etc. It may also be detected optically, with electrical switches, or via mechanical linkages or devices linked to one or more components associated with delivery of a dose.

The deterrent is a device which, once set, prevents, or at least discourages or inhibits delivery of a second dose into the delivery path, while the first dose is still also in the delivery path. The deterrent may be mechanical or electrical. The specific form of the deterrent, as well as the sensing device, is immaterial to the methods described. Once the deterrent is set or engaged, no further dose may be released or delivered into the delivery path. The deterrent is provided with an inhalation input indicating whether or when the first dose is inhaled. This input may be electrical, mechanical, pneumatic or magnetic, the specific form again not material to performance of the methods.

When the patient inhales the first dose, the inhalation input is triggered or switched on, at Step 4. This releases or resets the deterrent at Step 5. The dose delivery disabling or locking function of the deterrent is then deactivated or switched off. A second dose can then be delivered into the delivery path as shown at Step 6. The inhalation input may be designed to be triggered based on various parameters, including, for example, inhalation flow-rates (peak, averaged or other functions), vacuum, time or duration, volume, dose movement, etc. Generally, it is preferably triggered only when a large fraction, or a majority, or when substantially all of the dose, has been inhaled or has at least been moved out of the delivery path, as part of the dose inhalation process. The triggering parameters may vary with the pharmaceutical being delivered and potentially may also vary by patient.

This method of operation of an inhalation system helps to reduce the likelihood of a patient inadvertently delivering two or more doses into the delivery path, and then inhaling them. A patient may deliver a first dose into the delivery path, and then forget that a dose has been delivered. Or after the first dose is delivered into the delivery path, the patient may be interrupted, before inhaling the dose, causing the patient to be uncertain as to whether a dose has been delivered into the delivery path, or even inhaled. The method illustrated in FIG. 1 helps to reduce the potential of having a patient inhale more than one dose at a time in these and other settings.

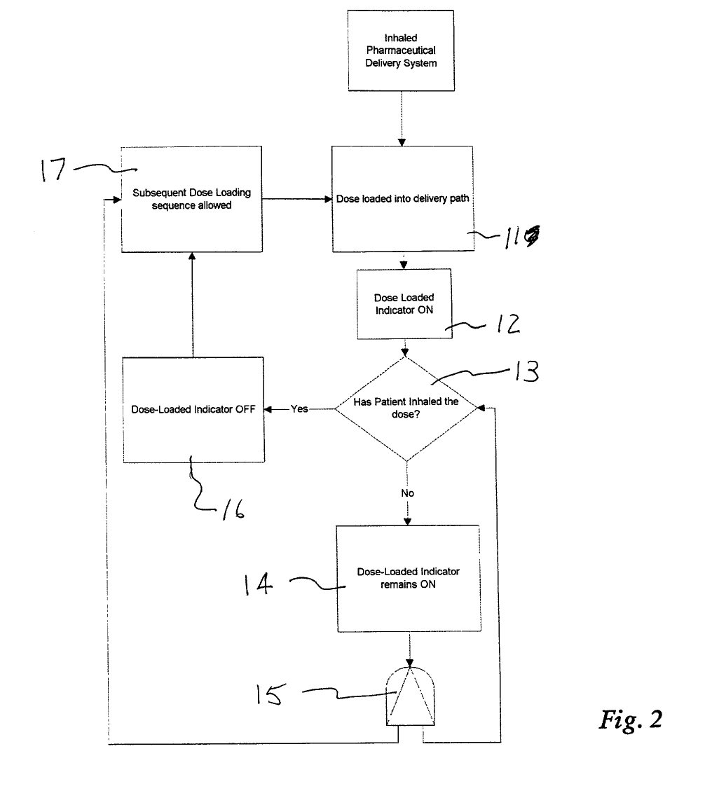

FIG. 2 shows a similar method. However, there is no actual automatic control of the releasing or delivering of the dose into the delivery path. Rather, an indicator is used to advise the patient that a dose has been delivered into the delivery path and that it should be inhaled before any further dose is delivered into the delivery path.

In FIG. 2, at Step 11, a dose is loaded into the delivery path, similar to Step 1 in FIG. 1. At Step 12, an indicator, typically a visual indicator such as a light or flag, is turned or set on. This provides a preferably visual indication that a dose has been delivered into the delivery path. The indicator may also provide a tone, sound or vibration, alone, or in combination with a visual indicator. If the indicator is a visual indicator, it is preferably on the front or top of the inhaler or otherwise in a location where it is easily viewed by the user. Step 13 in FIG. 2 is similar to Step 3 described above, i.e., monitoring to determine whether the dose has been inhaled. Again, this step can be performed mechanically or pneumatically, or via electrical or optical components. Once the dose is loaded, the indicator remains on, at Step 14, until the dose is inhaled. Once the system determines that the dose has been inhaled, the indicator is switched off at Step 16. This tells the patient that subsequent dose loading is now ready, at Step 17.

These methods can be used in virtually any inhaler intended to deliver one or a preset number of doses at a time, and where movement of a component is used to release or deliver a dose in preparation for inhaling the dose (regardless of the specific location in the inhaler). These methods are especially useful for dry powder inhalers of the type carrying multiple doses intended to be inhaled individually.

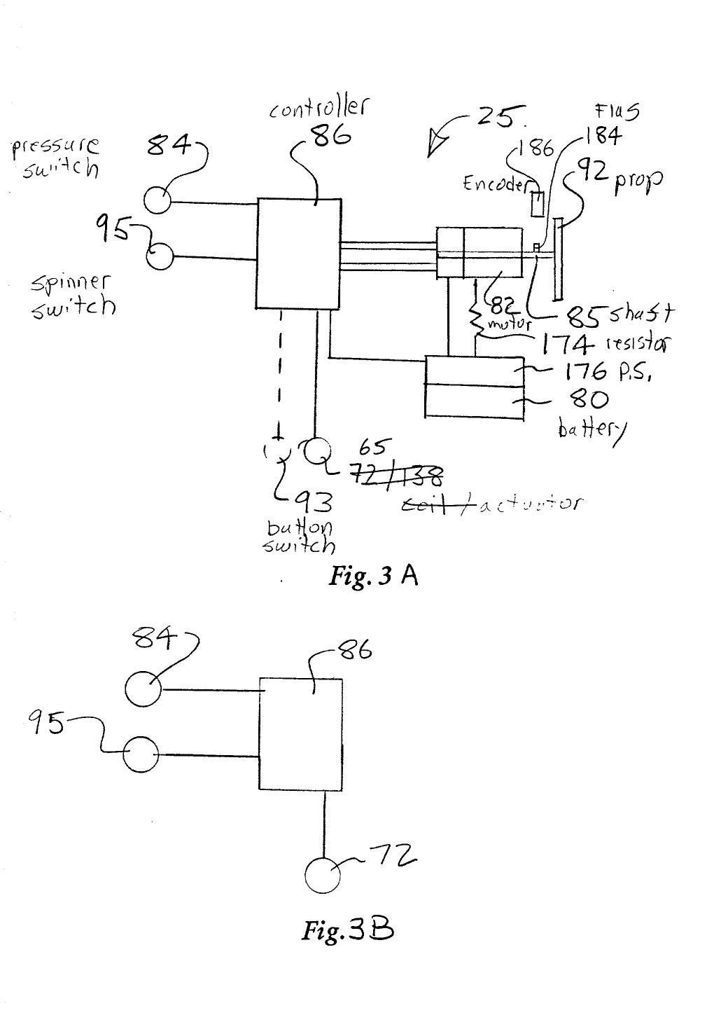

FIG. 3 schematically shows an inhaler 25 having a system for performing the methods shown in FIGS. 1 and 2. The inhaler may be similar to the inhalers shown in U.S. Pat. Nos. 5,492,112, 5,622,166, 5,921,237, 6,006,747, or 6,116,238, incorporated herein by reference. A propeller 92 is supported on a shaft 85 and spins within a chamber. An electric motor 82 drives the shaft 85. The motor 82 is powered via battery 80, a power supply 176, and a resistor 174. A controller 86, such as a microprocessor, is linked to the motor 82 and power supply 176. It is also linked to a pressure switch 84 and a delivery switch 95. The pressure switch 84 senses vacuum to detect inhalation. The delivery switch 95 detects movement of one or more components associated with delivery or release of a dose into a delivery path or other location. The controller 86 is also connected to an actuator 65. The actuator 65 provides for holding or movement of one or more components within the inhaler which largely prevent delivery or release of any subsequent dose.

A second delivery switch 93 may also be provided, depending on the dose release technique used in the inhaler. A flag or Hall effect device is attached to the shaft 85. An encoder is positioned near the flag and is linked to the controller.

In use, the patient activates the inhaler to deliver a dose into a delivery path. This movement is sensed by the controller 86 via the switch 96 and/or 94. The controller 86 then energizes the actuator 65, to mechanically lock the inhaler from delivering any subsequent dose. The inhaler remains in this locked condition until the patient inhales the dose in the delivery path.

When the patient inhales, the pressure switch 84 closes. The controller 86 turns on the power supply 176 to the motor 82, spinning up the shaft 85 and propeller 92. The controller 86 detects this inhalation either directly from the closing of the pressure switch 84, or via detection of shaft rpm speed via the encoder 186. If battery power is too low for continued use, the resulting slower shaft speed will be detected by the controller 86 via the encoder 186. The controller may optionally then leave the actuator in the locking position, to prevent further use until the batteries are replaced.

FIG. 3B shows a system 27 similar in design and operation to the system 25 shown in FIG. 3A, but without the motor 82, shaft 85, propeller 92, or encoder 186. This system 27 may be provided in inhalers which use patient inspiration to disperse the dose, such as the inhalers described in U.S. Pat. Nos. 4,811,731 and 5,829,434. The system 27 operates as described above, but without any of the motor related functions.

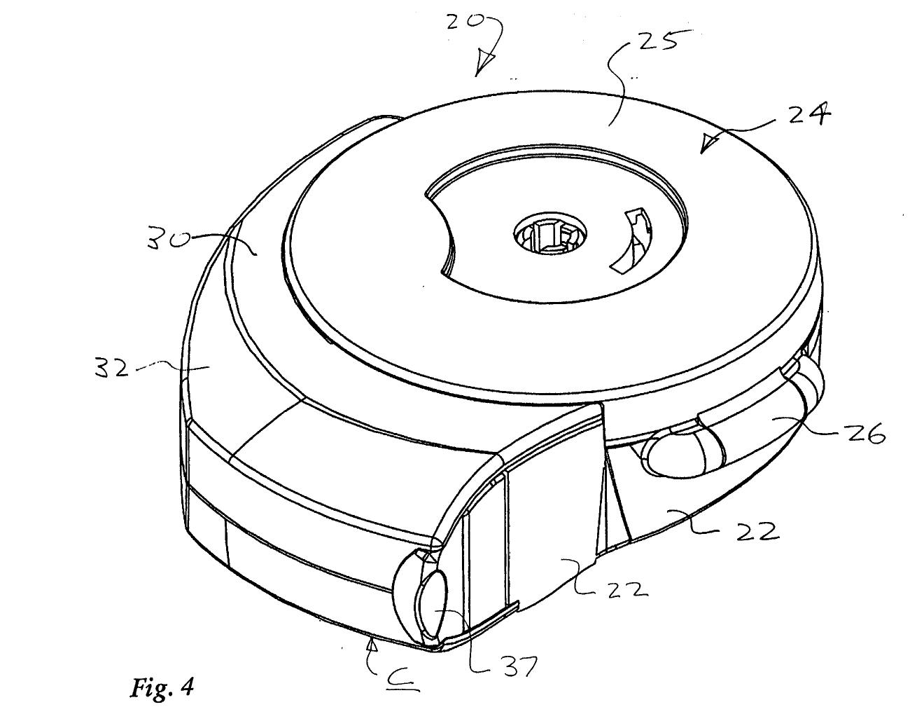

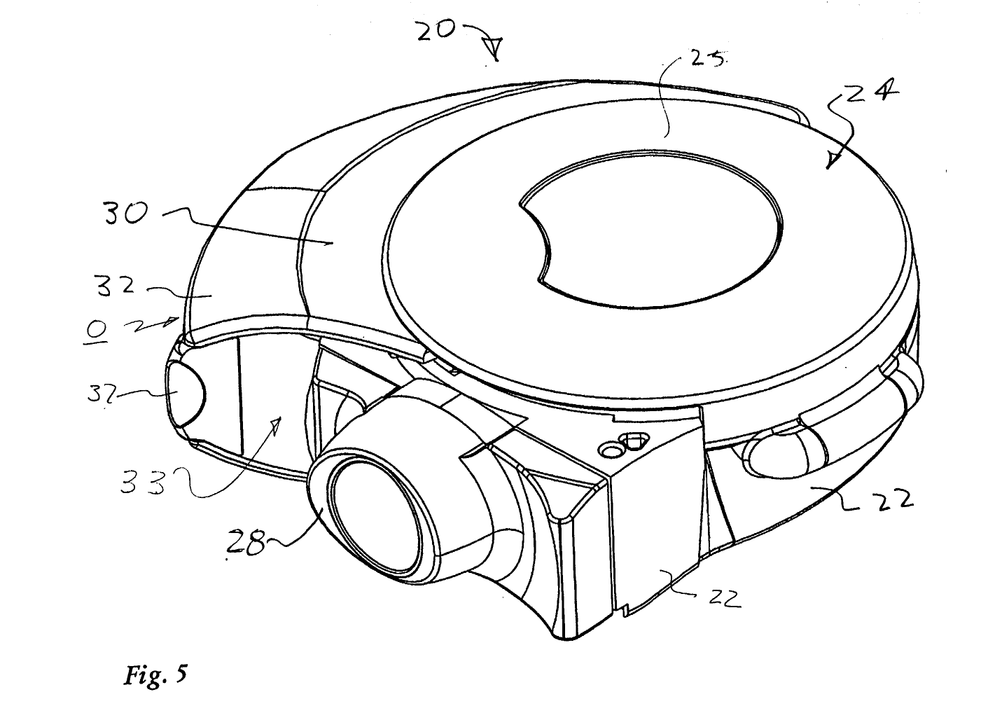

Referring to FIGS. 4-6, 5A and 5B, in an alternative embodiment, an inhaler 20 has a cover assembly 24 attached to a body or housing 22 via a hinge 26. A mouthpiece 28 is preferably attached to the front of the housing, and is removable for cleaning. A spinner assembly 30 is pivotably attached to a cover plate 25 on the cover assembly 24. The spinner assembly 30 includes an enclosure 32 having an open front 33. With the cover assembly 24 in the closed position, as shown in FIGS. 4-6, the spinner assembly 30 is pivotable from a closed position, as shown in FIG. 4, to an open position, as shown in FIGS. 5 and 6. With the spinner assembly 30 in the closed position, the enclosure 32 of the spinner assembly 30 covers the mouthpiece 28, as shown in FIG. 4.

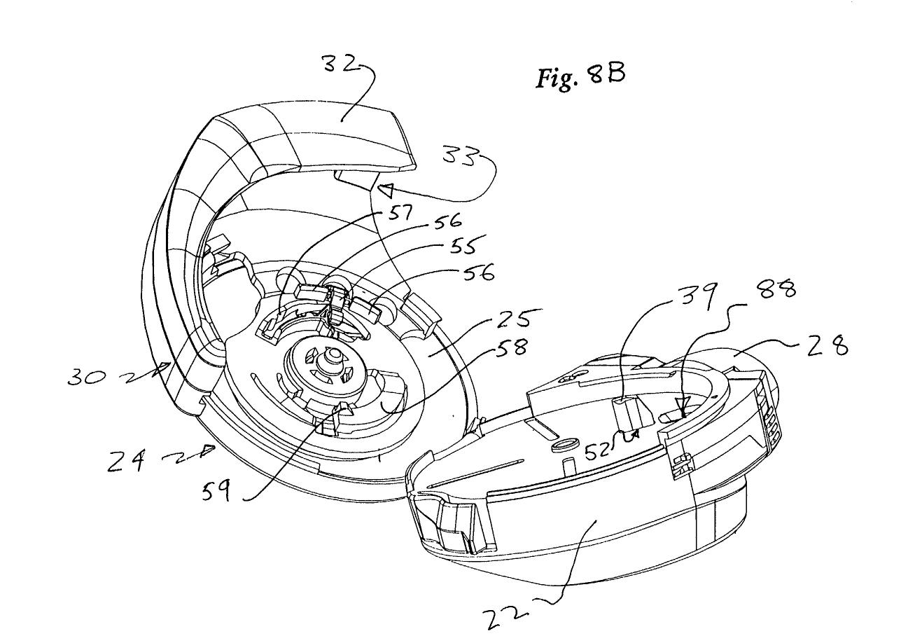

As shown in FIGS. 8A and 8B, the spinner assembly 30 includes a lever 55 pivotably supported on lever blocks 56 adjacent to a guide wall 57. A spring arm 58 includes an advancing tab 59. These features on the spinner assembly 30 operate to advance the blister disk 40, to deliver doses of pharmaceutical powder into the housing 22, as described in U.S. Pat. No. 5,921,237, incorporated herein by reference.

Referring to FIGS. 4-10, an actuation button 36 extends out from the bottom surface 34 of the inhaler housing 22. An actuation button shaft 38 attached to the actuation button 36 extends up through the housing 22, and with the upper end 39 of the actuation shaft 38 extendable out through a shaft opening 52 on the top deck 50 of the housing 22.

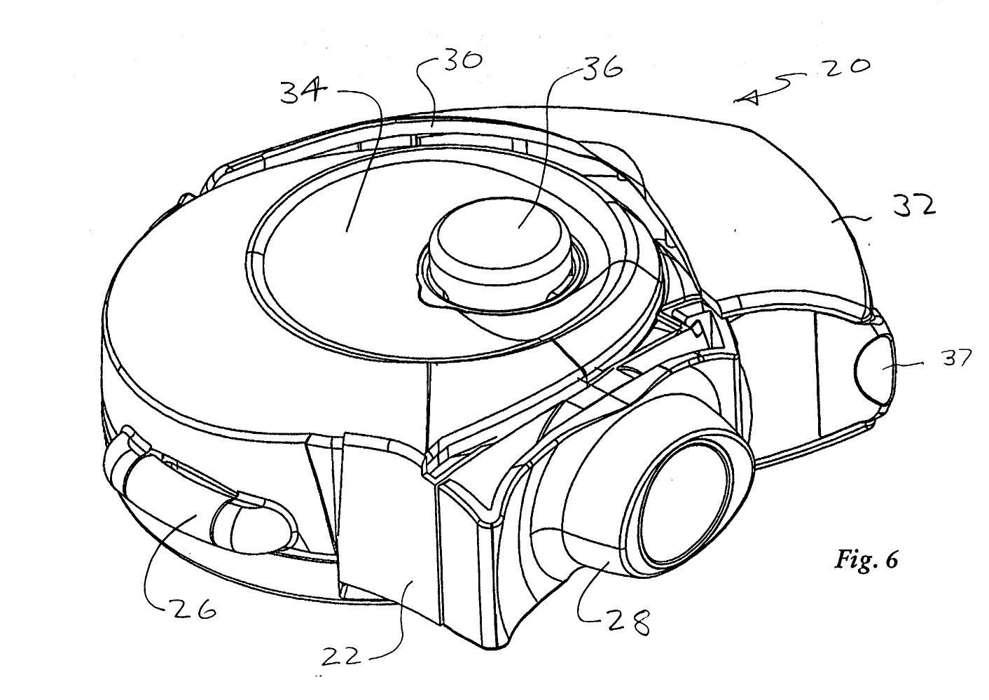

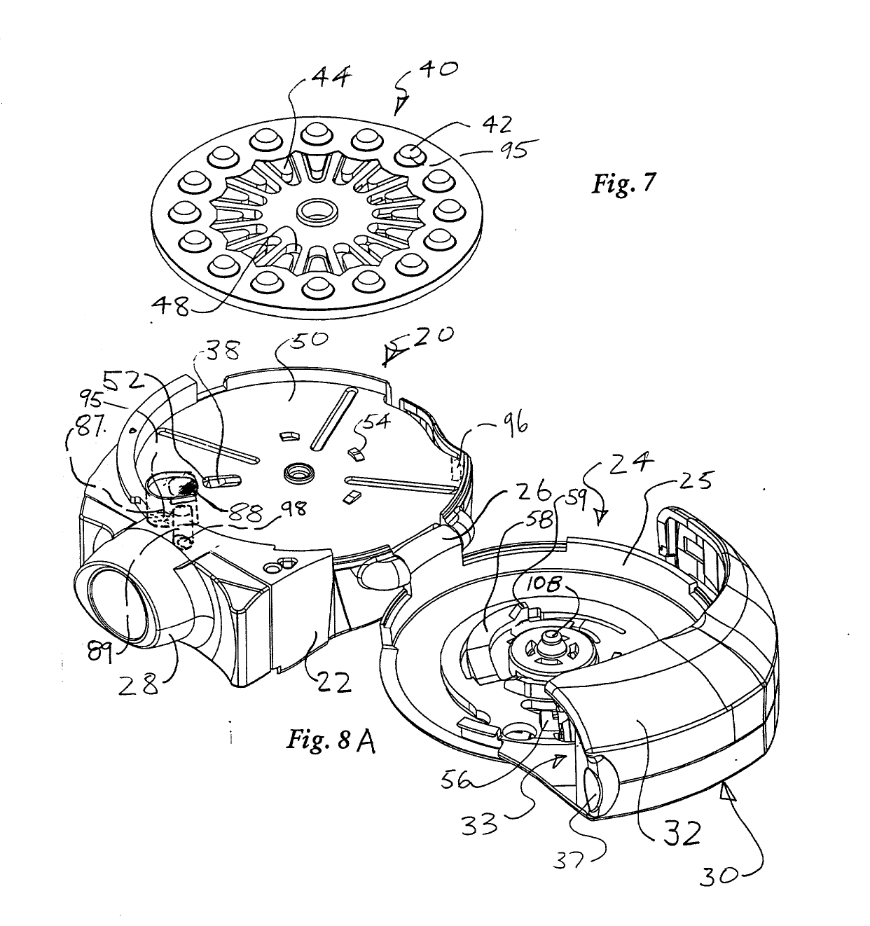

Referring momentarily to FIG. 7, the blister disk 40 has blisters 42 containing individual doses of a dry powder pharmaceutical. The blisters 42 are mounted on tabs 44 pivotable within tab slots 48 out of the plane of the blister disk 40, to shear out the bottom of the blisters 42, to release a dose of powder, as described e.g., in U.S. Pat. No. 5,622,166, incorporated herein by reference.

Anti-back rotation ramps 54 on the housing top deck 50 cooperate with the features on the spinner assembly 30 to prevent the blister disk 40 from moving in reverse, as the spinner assembly 30 is opened and closed.

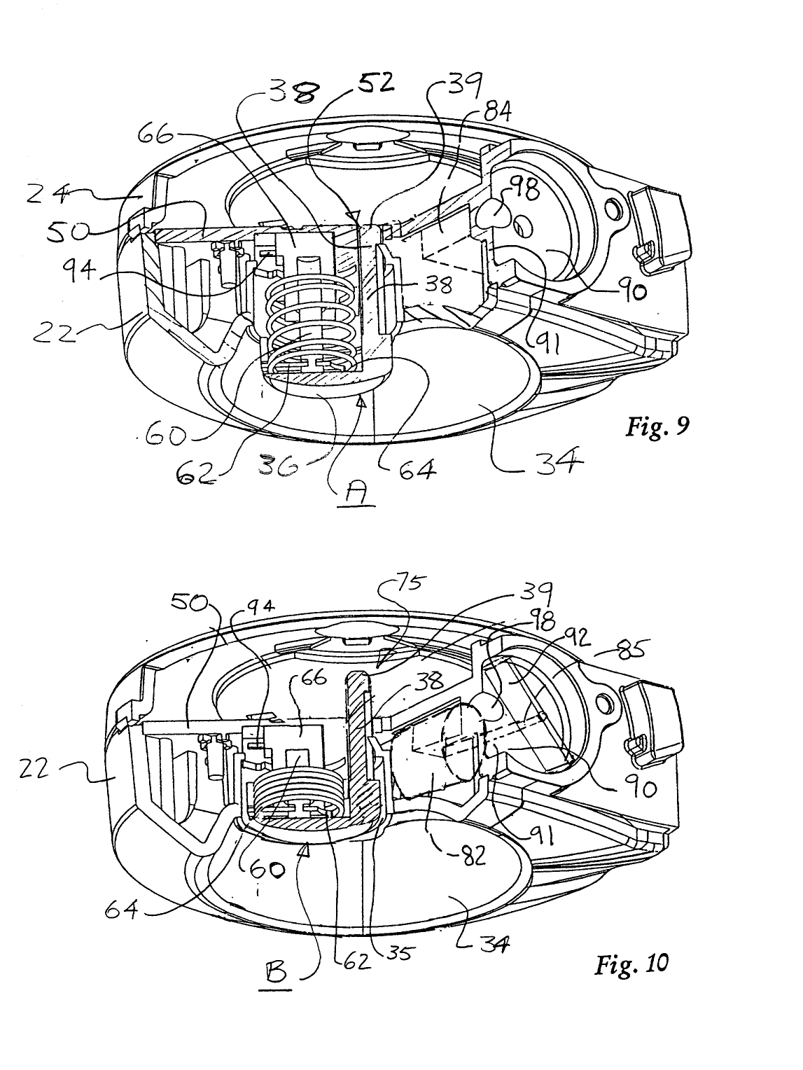

Referring to FIGS. 9, 10 and 11, a propeller 92 is attached to a motor shaft 85 for rotation within an aerosolizing chamber 90. An electric motor 82 is positioned within the housing 22 below the deck 50 and behind the rear wall 91 of the aerosolizing chamber 90. The motor and surrounding structure in FIG. 9 has been deleted for clarity of illustration.

The motor shaft 85 extends back through the rear wall 91 of the aerosolizing chamber 90 to an electric motor 82. Referring now also to FIG. 8A, a powder opening 88 is formed in the top deck 50 and extends downwardly into a powder chamber 87. A powder duct 89 extends between the powder chamber 87 and the aerosolizing chamber 90. The powder duct 89 opens into the aerosolizing chamber 90 via powder duct outlet 98 in the rear wall 91 of the aerosolizing chamber 90, as shown in FIGS. 8A and 10.

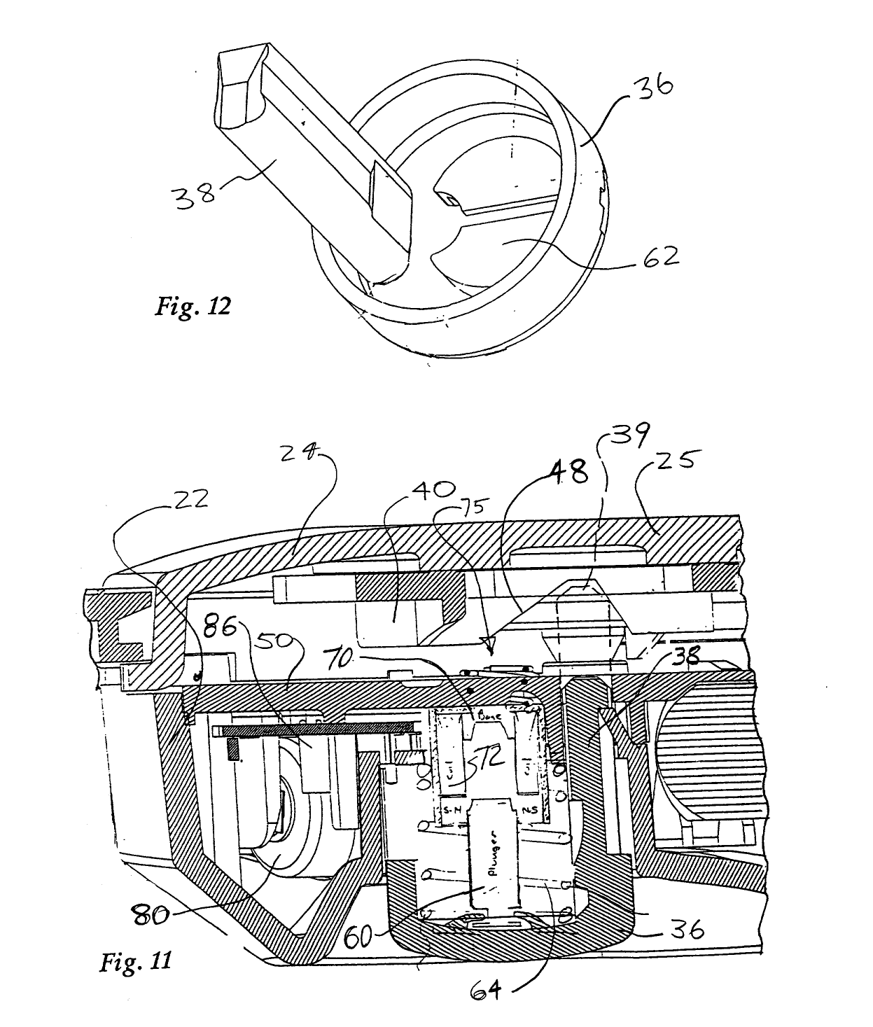

Turning now to FIGS. 8B, 9 and 10, the actuation button 36 is movable within a button cylinder 35 in the housing 22 from a first or down position A, as shown in FIG. 9, to a second or up position B, as shown in FIGS. 8B and 10. A plunger clip 62 formed on the top surface of the button 36 secures a plunger 60 onto the button. A compression spring 64 surrounding the plunger 60 biases the actuation button 36 away or outwardly from the housing 22, into position A. A permanent magnet/solenoid 66 is attached to the bottom surface of the deck 50 and surrounds the plunger 60. The plunger 60 is made of a magnetic material. Referring momentarily to FIG. 11, the permanent magnet/solenoid 66 includes a permanent base magnet 70 surrounded by coils 72. The permanent magnet/solenoid 66 may be used as an embodiment of the actuator 65 shown in FIG. 3A. Various equivalents of it may also be used.

A button switch 94 is adjacent to the actuation button 36 and detects whether the actuation button 36 is in the down (A) or the up (B) position. The button switch may be one embodiment of the switch 93 in FIG. 3A. A pressure or vacuum switch 84 is connected via a duct or passageway to the mouthpiece area or aerosolizing chamber 90 (or other flow passageway), to detect inhalation on the mouthpiece by a patient. A spinner switch 96 as shown in FIG. 8A, is located at the back of the inhaler housing 22, to detect whether the spinner is in the open position, as shown in FIG. 5, or in the closed position, as shown in FIG. 4. The spinner switch may be used as one embodiment of the switch 95 in FIG. 3A. One or more batteries 80 are included in the inhaler 20, to provide electrical power for the various electrical components in the inhaler. The electric components, including the motor 82, pressure switch 84, button switch 94, spinner switch 96, coil 72, (as well as other electrical components described below) are connected to an electronic controller or microprocessor 86.

In use, the cover assembly 24 of the inhaler 20 is opened, as shown in FIG. 8A, and a blister disk 40 is installed. The cover assembly 24 is then closed. With the spinner assembly 30 in the closed position, as shown in FIG. 4, the inhaler 20 may be carried by a patient, e.g., in a pocket or purse, ready for use as prescribed or needed. With the spinner assembly 30 in the closed position, the mouthpiece 28 is covered by the enclosure 32, to better keep the mouthpiece 28 clean.

To prepare the inhaler 20 for use, the patient moves the spinner assembly 30 from the closed position C shown in FIG. 1, to the open position 0 shown in FIG. 5. A spinner assembly release button 37 may be provided on the spinner assembly 30, to prevent the spinner assembly 30 from inadvertently being opened.

The patient next pushes the actuation button 36 up or in, from position A to position B, as shown in FIGS. 9 and 10. As this occurs, the upper end 39 of the actuation shaft 38 moves out of the shaft opening 52 in the top deck 50. The shaft end 39 pushes up on the tab 44 of the blister 42 aligned over the shaft opening 52. This causes the bottom of the blister 42 to shear open. The pharmaceutical powder in the blister 42 falls out of the blister 42, through the powder opening 88 in the top deck 50 and into the powder chamber 87. The upper end 39 of the shaft 38 of the actuation button 36 also pushes on the lever 55, on the spinner assembly 30, causing the lever to crush the blister 42 down, to assist in release of the powder from the blister 42 into the powder chamber 87.

Referring to FIGS. 9, 10, 11, as the actuation button 36 is moved from position A to position B, the permanent magnet 70 in the magnet/solenoid 66 attracts and holds the plunger 60, against the return force of the compression spring 64. As a result, the upper end 39 of the shaft 38 on the actuation button 36 remains engaged or extending into or through the tab slot 48 of the blister 42 aligned with the opening 52. This prevents the blister disk 40 from indexing or rotating to a next position. As the tab 59 on the spring arm 58 of the spinner assembly 30 is engaged with the blister disk 40, the spinner assembly 30 cannot be moved back to the closed position C until the actuation button 36 returns to position A.

Referring momentarily to FIG. 10, with the actuation button 36 in the up (B) position, the bottom surface of the actuation button 36 is pushed virtually entirely into the button cylinder 35 of the housing 22, so that the bottom end of the actuation button 36 is close to or flush with the bottom surface 34 of the housing 22. This prevents the patient from trying to pull the actuation button 36 out, because there is no exposed surface on the actuation button 36 to grab.

The inability of the patient to close the spinner assembly 30 indicates to the patient that a dose of powder 95 remains in the powder chamber 87, waiting to be inhaled. Thus, if the patient presses the actuation button 36, thereby releasing the dose of powder 95, the patient is reminded to inhale the dose, via the spinner assembly 30 remaining locked into the open position.

When the patient proceeds to inhale the dose of powder 95, by inhaling on the mouthpiece 28, the pressure switch 84 switches on the motor 82 which spins the propeller 92. The powder 95 is drawn through the powder duct 89 and into the aerosolizing chamber 90 via the patient's inspiration. The already rapidly spinning propeller 92 mixes and de-agglomerates the powder 95. The mixture of air and de-agglomerated powder passes out through the mouthpiece 28 and into the patient's lungs.

The controller 86 senses the inhalation detected by the pressure switch 84. Simultaneously, or after a short delay, the controller 86 energizes the coil 72. The current flowing through the coil 72 partially cancels out the magnetic field of the permanent magnet 70, sufficiently to allow the force of the compression spring 64 to drive the actuation button 36 down, from position B back to position A. This causes the actuation shaft 38 to be withdrawn back into the shaft opening 52, so that it no longer engages the blister disk 40 or any part of the spinner assembly 30. The blister disk 40 and spinner assembly 30 are then free to move. The patient can then move the spinner assembly 30 back to the closed position C, as shown in FIG. 4. As this occurs, the arm 58 and tab 59 on the spinner assembly 30 move the blister disk 40 so that the next blister 42 is aligned over the powder opening 88 and the tab 44 of the next blister is aligned over the shaft opening 52.

The permanent magnet/solenoid 66, together with the other components described, therefore form a spinner assembly lock system 75, which physically prevents a patient from unintentionally loading the pharmaceutical powder contents 95 from more than one blister 42 into the powder chamber 87, without inhaling.

As the coil 72 is only energized momentarily via the controller 86, the current drain on the battery 80 is minimal. With the plunger 60 in the down position A, the permanent magnet 70 is sufficiently distant from the plunger 60 to avoid any tendency for the permanent magnet 70 to inadvertently draw the actuation button 36 upwardly, without the patient pushing on the actuation button.

In another embodiment 100, as shown in FIGS. 10 and 11, the housing 22 has a deck latch 104 on a catch foot 102 at the back of the housing. The spinner assembly 110 has a spinner latch 112 adapted to engage with the deck latch 104.

In use, when the spinner assembly 110 is opened by the patient, the spinner latch 112 engages the deck latch 104, temporarily locking the spinner assembly 110 into the open position O. To return the spinner assembly 110 to the closed position C (as shown in FIG. 4), the patient must push in on the catch foot 102, to release the deck latch 104 from the spinner latch 112. While temporarily pushing in on the catch foot 102, the patient must also move the spinner assembly 110 at least slightly towards the closed position. After the deck latch 104 is disengaged and separated angularly from the spinner latch 112, the catch foot 102 may be released, and the pivoting movement of the spinner assembly 110 to the closed position C continued, until the spinner assembly 110 is moved fully into the closed position.

The inhaler 100 accordingly has a purely mechanical lockout which inhibits the patient from releasing more than one dose into the powder chamber 87, without inhaling. The extra step required to close the spinner assembly 110 (i.e., pushing in on the catch foot 102 to release the deck latch 104 from the spinner latch 112) helps to remind the patient to inhale the dose of powder 95 present in the powder chamber 87, before releasing a subsequent dose.

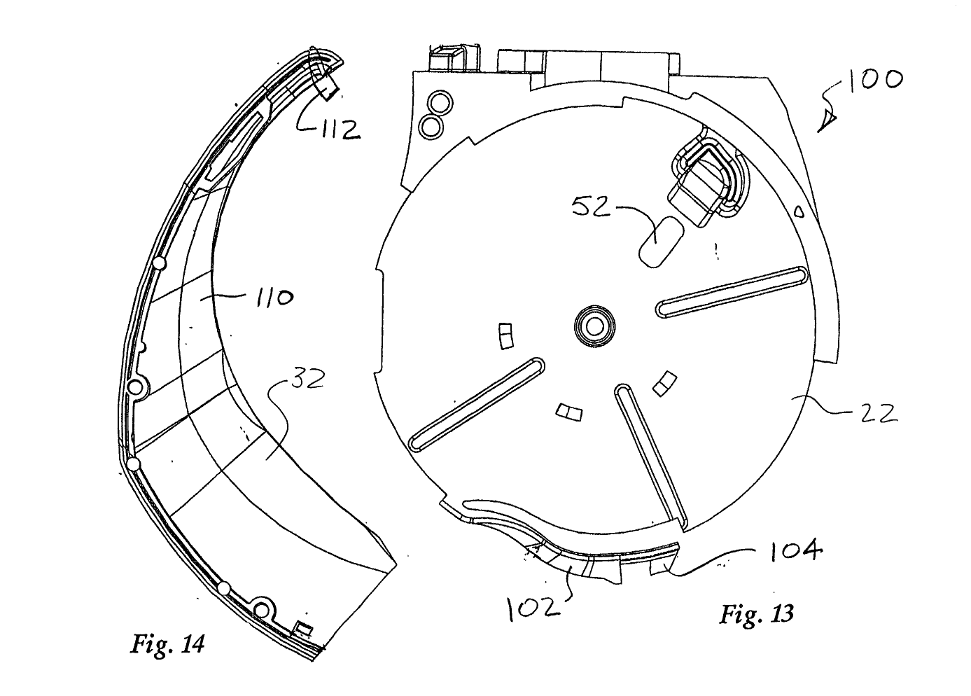

Referring to FIGS. 12 and 13, in another inhaler design 120, the inhaler housing 22 has a deck latch 104 on a catch foot 102, as in the inhaler 100 shown in FIGS. 10 and 11. Similarly, the spinner assembly 110 on the inhaler 120 has a spinner latch 112, as shown in FIG. 14. However, in addition to these features shared by the inhaler 100, the inhaler 120, as shown in FIGS. 12 and 13, has a lockout system 125, which prevents closing of the spinner assembly 110, unless the patient inhales between each dose released from the blister disk 40.

The lockout system 125 preferably includes a magnetic latching solenoid 122 having a solenoid plunger 124 attached to a sliding gate 126. A compression spring 127 biases the solenoid plunger and the sliding gate 126 outwardly, i.e., to the right side in FIG. 16. The sliding gate 126 has a blocking surface 130. With the sliding gate 126 in the extended position E as shown in FIG. 16, the blocking surface 130 prevents the catch foot 102 from being pressed in to release the deck latch 104 from the spinner latch 112. The blocking surface 130 on the sliding gate 126 is preferably supported by one or more gate guides 128 in the housing 22. The lockout system 125 provides a physical lock against closing the spinner assembly 110, with the lockout system switched on and off by the controller 86.

The magnetic latching solenoid 122 preferably includes a permanent magnet 134 which holds the solenoid plunger 124 into the R or retracted or in position, as shown in offset dotted lines in FIG. 16, for illustration purposes. The magnetic latching solenoid 122 preferably also includes a coil 138 around the permanent magnet 134. When energized with a positive polarity, the coil 138 cancels out enough of the permanent magnetic field of the permanent magnet 134 to allow the compression spring 127 to drive the plunger 124 and sliding gate 126 to the out or extended position E. A hard stop 136 limits the outward travel of the solenoid plunger 124 and sliding gate 126 to the position E where the blocking surface covers the recess 135. With the sliding gate 126 in this blocking position, the latches 104 and 112 cannot be released from each other, because the catch foot 102 cannot be pressed in towards the housing.

Conversely, applying negative or reverse polarity electric current to the coil 138 supplements the field of the permanent magnet 134, thereby increasing the magnetic attraction between the plunger 124 and the permanent magnet 134, and drawing the plunger 124 and sliding gate 126 into the in or R retracted position.

Electrical power is used by the lockout system 125 only to move the solenoid plunger 124 and sliding gate 126 between the R and E positions. No power is used to hold the plunger and sliding gate into these positions. The plunger and gate are held into the R position by the permanent magnet 134, and are held into the E position by the force of the spring 127.

In use, the patient installs a blister disk and opens the spinner assembly 110, as described above in connection with the other figures. When the spinner assembly 110 is moved into the open position, the spinner latch 112 engages and latches onto the deck latch 104. This prevents the spinner assembly 110 from being closed, without pushing in on the catch foot 102 to disengage the deck latch 104 and spinner latch 112. However, unlike the inhaler 110 shown in FIGS. 10 and 11, when the sliding gate is extended, the blocking surface 130 on the sliding gate 126 prevents the patient from pushing in on the catch foot 102 to release the deck latch 104 from the spinner latch 112, so that the spinner assembly 110 cannot be closed, until the sliding gate 126 is withdrawn. Specifically, the foot end 132 on the catch foot is prevented from moving into the foot end recess 135 in the housing 22.

The solenoid coil 138 is connected to the controller 86. With the solenoid 122 not energized, the gate 126, is held via the permanent magnet 134, into the retracted position R. The controller 86 monitors for signals from the spinner switch 96, button switch 94, and pressure switch 84, to detect when a dose of powder 95 has been released into the powder chamber 87, and inhaled by the patient. Upon release of a dose of powder, as detected by the closing of the button switch 94, the controller energizes the coil 138, partially canceling the field of the permanent magnet 134, and allowing the spring 127 to drive the gate into the extended position E. The patient now cannot close the inhaler for transport or storage, or to deliver another dose. Specifically, the patient cannot move the spinner assembly into the closed position, because the gate prevents release of the deck latch and spinner latch, even if the patient pushes in hard on the catch foot.

Upon detection of inhalation via a signal from the pressure switch 84, the controller 86, preferably after a short delay, momentarily energizes the solenoid 122 with reverse polarity, thereby adding to the field of the permanent magnet, and pulling solenoid plunger 124 and the sliding gate 126 into position R. With the sliding gate 126 withdrawn into position R, the blocking surface 130 is removed away from the foot end 132. The patient can then push the catch foot 102 in towards the housing 22, to release the deck latch 104 from the spinner latch 112.

As the patient pushes in the catch foot 102 and turns the spinner assembly, to move it towards the closed position, the controller 86 detects this movement via the spinner switch 96, and then de-energizes the solenoid 122. The sliding gate 126 and solenoid plunger 124 then return to the extended position E, via the force exerted by the spring 127. As the deck latch 104 and spinner latch 112 are already separated, the return movement of the sliding gate 126 does not further affect closing the spinner assembly 110.

The inhalers 20 and 120, via the switches or sensors 84, 94 and 92 and the controller 86, inhibit or prevent a patient from inadvertently inhaling a double dose or multiple dose, in a single open/closing/inhalation cycle. The inhalers 20 and 120 can also be designed to largely avoid having a patient inhale on the mouthpiece while thinking that a dose is being delivered, when actually no dose has been delivered, because the patient has forgotten to push the actuator button. The inhalers 20 and 120 provide a noticeable tactile sensation to the patient when the motor is on and the propeller is spinning. To better avoid missed doses due to other patients failure to push the actuation button and release a dose of powder, the controller 86 is preferably programmed to prevent turning on the motor 82, unless the controller detects release of a dose via the button switch 94. The failure the motor to turn on then provides a negative tactile indication to the patient to check for proper inhaler operation.

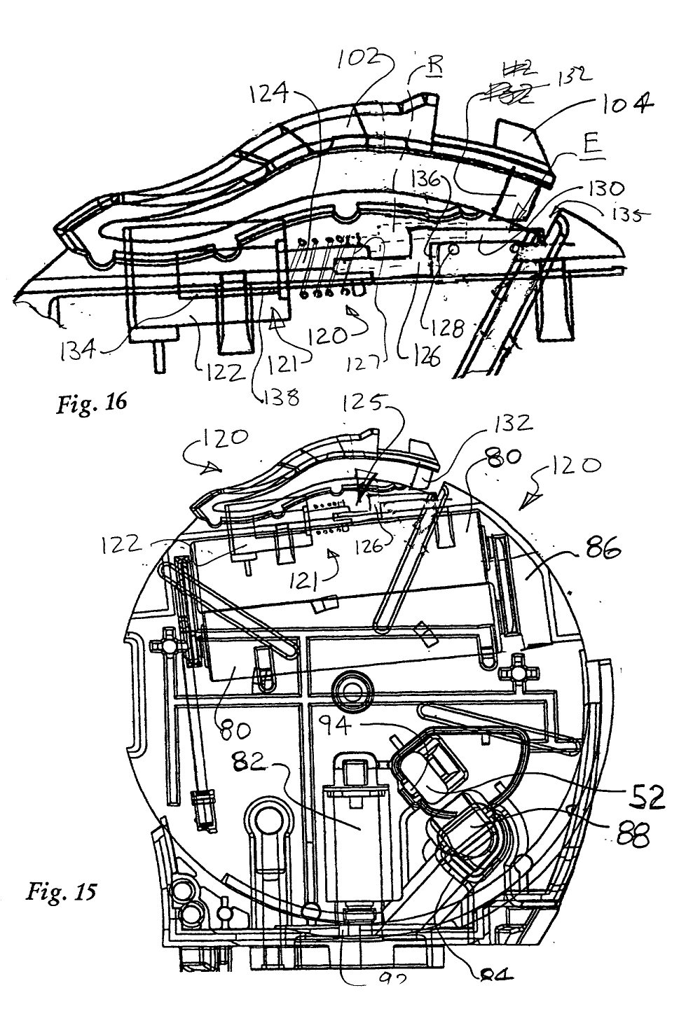

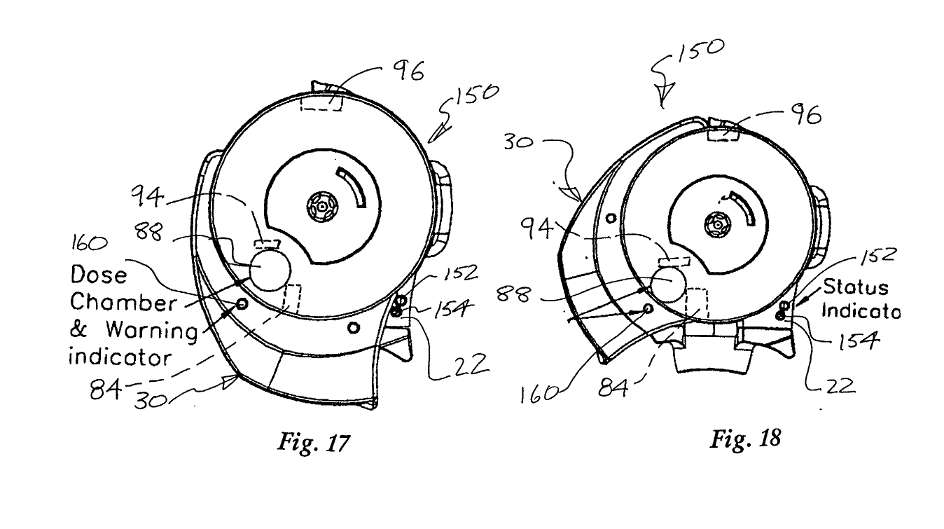

In a another design 150, shown in FIGS. 14 and 15, a visual indication system 151 is provided, to provide a visual indication to the patient that there may be more than one dose of powder 95 in the powder chamber 87. The visual indication system 151 intends to prevent, through notification, inadvertent inhalation of the more than one dose at a time. As shown in FIGS. 14 and 15, status lights, such as LEDs 152 and 154, preferably of varying colors, are provided on the housing 22 to provide indications of battery power, inhaler remaining life, etc. to the patient. In addition, a dose chamber warning light 160 is also provided on the housing.

The warning light 160 (as well as the other light indicators 152 and 154), are electrically connected to the controller 86. When the controller detects release of a powder dose into the powder chamber 87, via a signal from the button switch 94, the controller 86 causes the warning light 160 to light up. This indicates to the patient that a dose of powder is in the chamber, and should be inhaled before the spinner assembly is closed, or before the patient attempts to deliver another dose into the powder chamber 87 via movement of the spinner assembly 30. The controller 86 includes software which uses inputs from the pressure switch 84, the button switch 94 and the spinner switch 96. Using these inputs, the controller 86, via software logic, determines whether or not the patient has inhaled after the patient last pressed the actuation button 36, thereby releasing a dose of powder into the powder chamber 87. If the controller 86 determines that the patient has not inhaled the released dose, the warning light 160 lights up to indicate that a dose still remains in the chamber. The controller 86 may also optionally temporarily de-activate the motor 82, until the dose is cleared.

The warning state indicated by the warning light 160 can be cleared or resolved, to reset in the inhaler 150 back to normal operation in several alternative ways. It can be cleared by pressing the spinner and actuation button 36 simultaneously; by inhaling on the mouthpiece causing the pressure switch 84 to close; or by another sequence or combination of switch closures. The visual indication warning system 151 of the inhaler 150 thereby provides a visual indication of a potential multiple dose condition.

Thus, novel inhaler devices and systems have been shown and described. Various modifications may of course be made, without departing from the spirit and scope of the invention. The invention, therefore, should not be restricted or limited, except by the following claims and their equivalents.

1. A method for using an inhaler, comprising the steps of:
loading a dose of a pharmaceutical into a delivery path within the inhaler;
engaging a deterrent in the inhaler to deter loading of a subsequent dose;
detecting inhalation of the dose; and
disengaging the deterrent.

2. The method of claim 1 further including the step of loading a subsequent dose into the delivery path after disengaging the deterrent.

3. The method of claim 1 where the dose is loaded by moving a component within the inhaler, and the deterrent deters loading of a subsequent dose by at least indirectly restricting movement of the component, when the deterrent is engaged.

4. The method of claim 1 further including the step of engaging the deterrent by providing electrical power to the deterrent.

5. The method of claim 1 further including the step of providing a visual indication when the dose has been loaded into the delivery path.

6. An inhaler for providing multiple separate doses of a pharmaceutical, comprising:
a delivery path;
first means for determining whether a first dose has been released into the delivery path;
second means for determining whether a first dose released into the delivery path has been inhaled; and
means for deterring release of a second dose into the delivery path unless the first dose has been inhaled.

7. The inhaler of claim 6 where the delivery path comprises a space within an inhaler housing for temporarily holding a dose released from a blister.

8. The inhaler of claim 6 where the first means for determining comprises a switch actuated by movement of a component within the inhaler associated with releasing a dose into the flow path.

9. The inhaler of claim 6 where the second means comprises a pressure switch.

10. The inhaler of claim 6 where the means for deterring comprises an electrically powered actuator.

11. A dry powder inhaler for providing individual doses of a dry powder pharmaceutical formulation from a blister disk, comprising:
an inhaler housing;
a blister disk advancing mechanism for incrementally advancing the blister disk;
an actuator in the housing moveable from a first position, where the actuator does not engage the blister disk advancing mechanism, to a second position where the actuator engages and prevents advancing movement of the blister disk advancing mechanism;
an actuator holder in the housing adapted to hold the actuator in the second position; and
an actuator holder release which disables the actuator holder to allow the actuator to move from the second position to the first position.

12. The inhaler of claim 11 where the actuator holder comprises an electromagnet.

13. The inhaler of claim 11 where the actuator holder comprises a permanent magnet and an electromagnet.

14. The inhaler of claim 11 with the inhaler further including a breath actuated switch, and the actuator holder release includes a detector linked to the breath actuated switch.

15. The inhaler of claim 11 further including a mouthpiece and a powder port on the inhaler housing, with the blister disk advancing mechanism comprising a spinner assembly pivotably attached to a cover assembly on the housing, with the spinner assembly pivotable on the cover assembly from a closed position, where the spinner assembly covers the mouthpiece, to an open position, where spinner assembly is moved away from and exposes the mouthpiece, and where movement of the spinner assembly from the closed position to the open position does not move the blister disk, and where movement of the spinner assembly from the open position to the closed position moves the blister disk to position another blister on the blister disk over a powder port in the housing.

16. The inhaler of claim 11 further comprising an electronic controller in the housing and linked to the actuator holder and the actuator holder release.

17. A dry powder inhaler for providing individual doses of a dry powder pharmaceutical formulation from a blister disk, comprising:
a housing;
a cover assembly on the housing;
a spinner assembly pivotably attached to the cover assembly and including a blister disk advancer which advances the blister disk by one blister position, as the spinner assembly is moved between an open position and a closed position on the cover assembly;
an actuator in the housing moveable from a first position, where the actuator does not engage the spinner assembly, so that the spinner assembly is free to move, to a second position where the actuator engages the spinner assembly and prevents it from moving;
an actuator holder in the housing for holding the actuator in the first position until released;
an actuator holder release linked to the actuator holder for releasing the actuator holder;
an actuator sensor in the housing positioned to sense the position of the actuator;
an inhalation sensor in the housing positioned to sense inhalation; and
an electronic controller linked to the actuator holder release, to the actuator sensor, and to the inhalation sensor.

18. The inhaler of claim 17 where the actuator holder comprises a permanent magnet and the actuator holder release comprises a coil positioned to at least partially cancel the force of the permanent magnet when electric current passes through the coil.

19. A dry powder inhaler for providing individual doses of a dry powder pharmaceutical formulation from a blister disk, comprising:
a housing;
a cover assembly on the housing;
a spinner assembly pivotably attached to the cover movable between an open position and a closed position;
an actuator in the housing moveable from a first position, where the actuator does not engage the blister disk, so that the blister disk is free to advance, to a second position where the actuator engages the blister disk and prevents it from advancing;
an actuator holder in the housing for holding the actuator in the first position until released; and
an actuator holder release associated with the actuator holder.

20. A dry powder inhaler for providing individual doses of a dry powder pharmaceutical formulation from a blister disk, comprising:
a housing;
a cover assembly on the housing;
a spinner assembly pivotably attached to the cover assembly and including a blister disk advancer for engaging and advancing the blister disk as the spinner assembly is moved from an open position to a closed position on the cover assembly;
a housing latch on the housing; and
a spinner assembly latch on the spinner assembly engagable with the housing latch, for locking the spinner assembly into the open position until the housing latch is disengaged from the spinner assembly latch.

21. The inhaler of claim 20 further including a catch foot resiliently attached to the housing and with the housing latch located on the catch foot.

22. A dry powder inhaler for providing individual doses of a dry powder pharmaceutical formulation from a blister disk, comprising:
a housing;
a cover assembly on the housing;
a spinner assembly pivotably attached to the cover assembly and including a blister disk advancer for engaging and advancing the blister disk as the spinner assembly is moved from an open position to a closed position;
a housing latch on the housing;
a spinner assembly latch on the spinner assembly engagable with the housing latch;
a gate movable from a first position where the gate prevents disengagement of the housing latch and the spinner latch assembly, to a second position where gate allows disengagement of housing latch and spinner latch assembly; and
a gate actuator attached to the gate.

23. The inhaler of claim 22 where the gate and the gate actuator are in the housing.

24. The inhaler of claim 23 where the gate actuator comprises a permanent magnet latching solenoid.

25. The inhaler of claim 23 further comprising a breath inhalation detector in the housing for detecting inhalation by a patient using the inhaler, an actuator detector in the housing positioned to detect movement or position of the actuator, and an electronic controller linked to the inhalation detector, the actuator detector, and to the gate actuator.

26. A method for providing individual doses of a dry powder pharmaceutical formulation from a blister disk, comprising the steps of:
pivoting a spinner assembly on an inhaler from a closed position to an open position;
moving an actuator in the inhaler from a first position to a second position so that the actuator at least indirectly opens a blister on the blister disk, and so that the actuator prevents advancing movement of at least one of the spinner assembly and the blister disk;
holding the actuator in the second position;
detecting inhalation by a patient using the inhaler;
releasing the actuator and allowing the actuator to return to the first position upon detection of inhalation; and
pivoting the spinner assembly back to the closed position.

27. An inhaler comprising:
a dose release mechanism;
a dose release sensor;
an inhalation sensor;
an indicator; and
a controller connected to the dose release sensor, the inhalation sensor, and the indicator, with the controller causing the indicator to switch on when a dose release is sensed by the dose release sensor, and to thereafter switch off the indicator upon inhalation sensed by the inhalation sensor.

28. The inhaler of claim 27 where the indicator comprises a visual indicator.

29. The inhaler of claim 27 where the dose release sensor comprises a switch associated with an actuation button used to release a dose of a pharmaceutical for inhalation.
